(19) United States
(12) Patent Application Publication
(10) Pub. No.: US 2022/0226188 A1
(43) Pub. Date: Jul. 21, 2022

(54) VIBRATION GENERATOR

(71) Applicant: SynDermix AG, Stans (CH)

(72) Inventors: Carlos Roberto CAMOZZI, Basel (CH); Samuel MALZACH, Evilard (CH); Steve KNUCHEL, Lyss (CH)

(21) Appl. No.: 17/596,093

(22) PCT Filed: Jun. 8, 2020
(86) PCT No.: PCT/EP2020/065867

§ 371 Date: Dec. 2, 2021

(30) Foreign Application Priority Data
Jun. 7, 2019 (CH) 00781/19

(51) Int. Cl. A61H 23/02; H04R 9/06; H04R 9/02; H04R 9/04; H04R 3/00; B06B 1/04

(52) U.S. Cl. CPC A61H 23/0236; H04R 9/06; H04R 9/025; H04R 9/022; H04R 9/045; H04R 3/00; B06B 2201/76; A61H 2201/0153; A61H 2201/123; A61H 2201/1685; A61H 2201/5007; A61H 2205/02; B06B 1/045

(57) ABSTRACT

The invention relates to the field of transducers, in particular linear vibration generators. It concerns a vibration generator, devices comprising such a vibration generator and a related treatment method.

A vibration generator comprising a mass, a coil, a permanent magnet and a housing, wherein the mass can be set in an oscillatory motion with respect to the housing by applying a current to the coil, and the vibration generator further comprising an axle, wherein the oscillatory motion is along the axle, and in that the mass comprises the permanent magnet and the coil is fixed to the housing.

### FIELD

The invention relates to the field of transducers. It concerns a vibration generator, in particular a linear vibration generator. The vibration generator is in particular—but not exclusively—suitable for devices for medical applications, in particular medical devices for stimulation of a subject by oscillations (vibrations), in particular vibration therapy such as modulated vibration therapy. Sound-vibrational therapy, therapy by acoustic energy, therapy by ultrasound or therapy by infrasound are examples of (modulated as the case may be) vibration therapies. The invention relates further to a device for oscillation (vibration) therapy comprising the vibration generator and to a related method.

### BACKGROUND

Linear vibration generators comprise a mass that can be actuated to carry out an oscillatory motion along an axis. Therefore, the vibration generator comprises a permanent magnet or an electromagnet, a coil, and means for generating a repelling force.

There are various kinds of linear vibration generators known. In consumer electronics, a linear vibration generator usually comprises a permanent magnet or an electromagnet that is firmly mounted to a hosing of the vibration generator and a coil that is firmly mounted to the mass.

State-of-the-art linear vibration generators are rather complex in terms of number of parts and design. US 2019/0068039 A1 and WO 2011/043536 A1 show example of state-of-the-art vibration generator. Often, they work at resonance frequencies of the oscillatory motion in order to be efficient and to have a high response sensitivity and/or they are optimized for a narrow frequency range. WO 00/058505 A1 discloses a vibrator optimized for the very low frequency range, this means for the frequency range below 30 Hz.

Devices for oscillation (vibration) therapy are well-known in the exemplary field of medical applications.

For example, WO 2011/159317 A1 discloses a pain abatement device that provides for multiple sensory inputs, wherein the multiple sensory inputs are generated by temperature, a tactile input and vibration by utilizing multiple small vibratory motors.

US 2012/0253236 A1 discloses wearable devices for externally delivering therapeutic stimulation to improve health, condition and performance. The stimulation is done via vibration, tones, audio or electrical pulse, light or other sources. In embodiments, the device comprises a regular or vibration speaker or a vibrating component with a motor.

US 2003/0172939 A1 discloses a method and a device to relieve discomfort by attaching a vibration generating means to hard tissue of the patient's head and by applying vibrations at a subsonic frequency.

US 2008/0200848 A1 discloses a method and a device for treating nasal congestion and/or relieving sinusitis symptoms, in particular by combining vibrational stimulation and a stream of fluid forced towards the patient's respiration tracks.

US 2013/0253387 A1 discloses systems and methods for treating an occluded area in a body or for reducing pathologic material in the body, for example. Therefore, vibratory energy is applied to pathologic material in a treatment area of the body. The vibratory energy is provided to the treatment area by use of a piezoelectric transducer and an effector, wherein the effector can be designed to reach into the occluded area or to be positioned on a forehead or another external body portion.

WO 2010/113046 A1 discloses a device for the ventilation of nitric oxide in the paranasal sinuses and to suppress disorders of the upper respiratory tract. The device comprises a vibration generator, a vibration transmitter in mechanical/physical contact with the vibration generator, and a control unit. The vibration generator contains an electric motor and an eccentric wheel. Control unit, vibration generator and vibration transmitter are designed to allow for a fast revolution changes in a given frequency range.

CN 108704 826 A describes a device having an axle i.e. guiding column (2) comprising two ends which are covered by a first and second fixing member (6, 10) upon which a first and second coil is wound (1, 4). A magnet is placed in the middle of the axle (2).

WO 2015/030602 A1 describes a vibrator apparatus for stochastic vibrations. The vibrator apparatus (34) comprises a moving coil (35) which is rigidly attached to a member (1″), e.g. a frame or beam, of a vibration device (1), the moving coil (35) for an elected vibration mode of the apparatus being configured to receiving electric signals, e.g. pulsating and/or sinusoidal electric signals from a signal unit (25), and the moving coil (35) co-operates with a permanent magnet (48) which is suspended by springs (49, 50; 93, 94) attached said member (1″), and the coil (35) and the magnet (48) are mutually linearly and coaxially movable upon application of said electric signals to the moving coil (35). The stated purpose of the apparatus is to provide external stochastic noise having allegedly positive effects on for example hearing, ADHD and dopamine-related neurodegenerative disturbances, such as akinesia, Parkinson and aging.

U.S. Pat. No. 3,366,749 A describes an audio transducer having an axle i.e. moving post 6 having a rod 10, a voice coil 16 and a permanent magnet 8 both of which surround the axle. Resilient mounting means 9 appears to hold the magnetic assembly and is attached to the axle.

DE 10 2015 209639 A1 is related to mobile phone technology and describes an electromagnetic linear actuator creating a haptic output. The actuator has an axle i.e. shaft 320, a moving mass/central magnet array 310 and a coil 300.

JP 2005 348815 A describes a vibrating massage apparatus for the human body for fatigue recovery and blood circulation improvements. The apparatus has a mass that extends from a spring member but an axle does not appear to be present.

State-of-the-art vibration generators are not suitable for many applications, in particular medical applications. Their design cannot be adjusted easily to different frequency ranges and/or their frequency tuning properties are limited and/or far from being linear. Further, the impulse that can be generated is not sufficient for many applications for example medical applications.

### SUMMARY OF THE INVENTION

In a first aspect of the invention there is provided a vibration generator comprising a mass, a coil, a permanent magnet and a housing, wherein the mass can be set in an oscillatory motion with respect to the housing by applying a current to the coil, and the vibration generator further comprising an axle, wherein the oscillatory motion is along the axle, and in that the mass comprises the permanent magnet and the coil is fixed to the housing.

In embodiments the permanent magnet is a ring magnet, wherein the ring magnet and the coil are arranged concentrically around the axle.

In a further embodiment the mass comprises a slit, wherein the slit is arranged concentrically with respect to the axle and wherein the coil is arranged in the slit.

In another embodiment the mass and the permanent magnet are configured to generate an essentially homogeneous field in a section of the slit, wherein the homogeneous field runs radial to the axle in this section of the slit.

In embodiments the section is formed between a core ring and a core bottom, wherein the core ring is of a material having a high saturation level and wherein the core bottom is configured to not exceed a saturation limit of the material having the high saturation level.

In a further embodiment an extension of the coil in a direction parallel to the axle is smaller than an extension of the section in a direction parallel to the axle and wherein the vibration generator is configured such that the coil is in the section independent of the orientation of the vibration generator.

In another embodiment the vibration generator is configured for the oscillatory motion being restricted between two positions of maximum deflection of the mass and wherein the vibration generator is configured for the coil being predominantly in the section of the homogeneous field.

In embodiments an extension of the coil in a direction parallel to the axle is larger than an extension of the section in a direction parallel to the axle, wherein the vibration generator is configured for the oscillatory motion being restricted between two positions of maximum deflection of the mass and wherein the vibration generator is configured for a portion of the coil extending over the full extension of the section independent of the position of the mass.

In a further embodiment the vibration generator comprises an elastic element that centers the mass when the vibration generator is not powered.

In another embodiment the elastic element is compressed during operation of the vibration generator.

In embodiments the vibration generator is configured to have its basic harmonic outside the frequency range in which the vibration generator is operated.

In a further embodiment the vibration generator is configured to have no harmonics of significance with respect to the amplitude of an oscillatory motion of the mass in the frequency range in which the vibration generator is operated.

In another embodiment the coil is mounted on a support having good heat transfer properties, wherein the support is in thermal connection to the housing, and wherein the housing is of a material capable to absorb heat generated by the coil and transferred to the housing via the support.

In embodiments the vibration generator further comprises a signal processing unit, wherein the signal processing unit is configured to superimpose a control signal used for the oscillatory motion of the mass with a further signal, wherein the further signal and the vibration generator are configured in a manner that an audible signal can be generated from the further signal by the vibration generator.

In a further embodiment the vibration generator is configured to sweep over a plurality of frequencies.

In another embodiment the vibration generator is configured to oscillate at a frequency of not less than 1 Hz, 5 Hz, 10 Hz, 20 Hz, 30 Hz, 40 Hz, 50 Hz, 60 Hz, 70 Hz, 80 Hz, 90 Hz, or 100 Hz.

In a further embodiment the vibration generator is configured to oscillate at a frequency of not more than about 2000 Hz, 1900 Hz, 1800 Hz, 1700 Hz, 1600 Hz, 1500 Hz, 1400 Hz, or 1300 Hz.

In embodiments the vibration generator is configured for oscillations in the range of 1 Hz to 2000 Hz, more suitably in the range of 20 Hz to 1500 Hz, and optionally in the range of about 60 Hz to about 1300 Hz.

In a further embodiment the vibration generator is configured to sweep over a frequency range of about 60 to about 1300 Hz, or a section thereof.

In another embodiment the sweep occurs over a time period of at most about 60 s, 45 s, 30 s, 25 s, 20 s, 15 s, 10 s, or 5 s.

In a further aspect there is provided a device for applying oscillations to a subject to be stimulated, comprising a vibration generator according to the preceding aspect and embodiments.

In a further embodiment the device comprises a device head and a device body, wherein the vibration generator is arranged in the device head.

In another embodiment the device head is movable to a first position relative to the device body and to a second position relative to the device body, wherein the device comprises a controller configured to switch the device in a sleeping mode if the device head is moved to the first position and to switch the device in an active mode, if the device head is moved to the second position.

In embodiments the device head is movable to a third position relative to the device body, wherein the third position allows access to a contact surface for cleaning and wherein the controller is configured to switch the device in the sleeping mode if the device head is moved to the third position.

In another aspect there is provided a method for treating a subject with oscillations, characterized by comprising a step of bringing a device comprising a vibration generator according to one of the above aspects and embodiments in contact with the subject.

### BRIEF DESCRIPTION OF THE DRAWINGS

FIG. 1 an exterior view of an exemplary embodiment of a device comprising a vibration generator;

FIG. 2 an external view of a further exemplary embodiment of a device comprising a vibration generator;

FIG. 3 an external view of yet a further exemplary embodiment of a device comprising a vibration generator;

FIG. 4 an exploded view of device shown in FIG. 1;

FIG. 5 an exploded view of an exemplary embodiment of the device head shown in FIG. 1;

FIG. 6 an exploded view of a further exemplary embodiment of a device head;

FIG. 7 a sectional view of the device head of FIG. 5;

FIG. 8 an exploded view of an exemplary embodiment of a vibration generator;

FIG. 9 a sectional view of the vibration generator of FIG. 8:

FIG. 10 a detail view of an actuation region of the vibration generator shown in FIG. 8;

FIG. 11 a detail view of an alternative embodiment of the actuation region; and

FIGS. 12-15 CRS treatment as an application example.

### DETAILED DESCRIPTION

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

Embodiments of the vibration generator and the device according to the invention are in particular suitable for vibration therapy, in particular modulated vibration therapy, that is applied to an exterior body portion.

In embodiments suitable for modulated vibration therapy, the frequency is modulated at least, for example by applying a sweep as disclosed below.

Vibration therapy is used for several medical applications such as chronic rhinosinusitis (CRS), migraine, chronic wound healing, pain relief, and muscular tension. There are indications of a potential use of vibration therapy in various further medical applications as pointed out below.

The main advantages of (exterior) vibration therapy over other therapy methods are its non-invasive, drug-free and safe character without significant loss of local applicability if directed vibrations (as provided by the vibration generator according to the invention) are used. Further advantages are easy and comfortable applicability if a treatment is carried out with a device according to the invention.

The specific biological, physical and chemical effects caused in a living body by vibration therapy are still being investigated in future trials, but the general effects are discussed in the following. The general effects of vibration therapy comprise vasodilatation, stimulation of cells, and enhancement of secretion clearance (for example by promoting transport and/or (out)flow) among others.

In the following, it is shown on the example of the treatment of chronic rhinosinusitis (CRS) how these effects cause a significant therapeutic effect. The vibration generator and device are in particular configured to cause at least one of these effects and hence to cause said therapeutic effect (as shown in the “application example” given below).

If a device for vibration therapy is applied on the cheekbone for the treatment of CRS (chronic rhinosinusitis), vibrations propagate to the paranasal sinuses like the maxillary sinus and to the nasal cavity and set the paranasal sinuses and the nasal cavity in oscillation. These oscillations accelerate the transport in the nose of excessive mucus and secretions, for example by mechanically induced transport and/or by increase of the mucociliary clearance, and stimulate the nasal and paranasal epithelium, for example by setting the epithelium in vibration and by vasodilatation. Both accelerated transport and stimulation accelerate the healing process, in particular reduce inflammation, and contribute to an opening of the ostium of the paranasal sinuses. The latter in combination with a vibrating maxillary sinus allows for a promptly release of nitric oxide (NO) from the paranasal sinuses into the nasal cavity. In addition, the vibration of the maxillary sinus presumably promotes NO production. There are indications that a high NO concentration has a protective or even healing effect, said effect being active in the maxillary sinus and nasal cavity due to the given mechanism of action.

In summary, vibration therapy enhances and accelerates the healing process, reduces the pathognomonic symptomatology of CRS (e.g. facial pain, congestion, rhinorrhoea, etc.) and improves the well-being of the patient with CRS both in the short and long-term. In other words, it shows anti-inflammatory, antioedematous and antiallergic effects, promotes normalisation of body defences, and may be used as monotherapy. The method is physiological, and it reduces the number of punctures in maxillary sinusitis, leaves the skin and mucosa intact, and decreases the use of drugs.

The mechanism of action summarized in the preceding paragraphs will be further explored using the vibration generator and device disclosed.

The effect of applying a vibration generator and a device as disclosed will be further explored in clinical tests. It is envisaged to evaluate at least one of the change in subjective symptoms as quantified by the German validated disease-specific 20-item Sino-nasal Outcome Test (SNOT-20 GAV), the change in endoscopic appearances, the change in need for surgical intervention, the change in the ability to perform normal activities, overall disease control, acceptability of treatment, overall score SNOT-20, pain score (VAS), and adverse events.

Vibration therapy in general has the potential for treating various medical conditions and reasons for physical uneasiness based on the biological, physical and chemical effects mentioned above and if the applied vibrations have characteristics suitable to cause these effects.

There are indications that vibration therapy increases angiogenesis and granulation tissue formation and reduces neutrophil accumulation and increases macrophage accumulation. Additionally, it may increase expression of pro-healing growth factors and chemokines (insulin-like growth factor-1 (IGF-1), vascular endothelial growth factor (VEGF) and monocyte chemotactic protein-1) in wounds (Eileen M. et al., 2014; PLoS ONE 9(3)). Vibration exposure may increase gene expression of collagen-1a (3-fold), IL-6 (7-fold), COX-2 (5-fold), and bone-morphogenetic-protein-12 (4-fold) (Thompson W et al., The Orthopaedic Journal of Sports Medicine, 3(5)).

It is an object of the invention to provide a linear vibration generator that overcomes drawbacks of state-of-the-art linear vibration generators.

In particular, it is an object of the invention to provide a linear vibration generator and a device comprising such a vibration generator that overcome drawbacks of state-of-the-art linear vibration generators and devices with respect to medical applications, in particular with respect to vibration therapy, such as modulated vibration therapy.

For example, it is an object of the invention to provide a linear vibration generator and a device suitable for the treatment of CRS by (external) vibration therapy, in particular modulated vibration therapy.

For example, it is an object of the invention to provide a linear vibration generator with improved frequency tuning properties. In particular, it is an object to provide a linear vibration generator comprising a mass that is configured to carry out an oscillatory motion, wherein the vibration generator is tunable over a frequency range of interest and wherein the amplitude of the oscillation is more homogeneous over the whole frequency range of interest compared to state-of-the-art linear vibration generators.

For example, it is an object of the invention to provide a linear vibration generator suitable for generating more powerful impulses compared to state-of-the-art linear vibration generators.

At least one of these objects is achieved by the devices and methods according to the claims.

In particular, the axle is firmly mounted to the housing. In other words, the axle does not move relative to the housing, but it is an axle of the oscillatory motion of the mass.

In particular, the axle is a straight axle.

The axle can define a (directional) axis that can be a longitudinal axis.

The mass comprises the permanent magnet and the coil is fixed to the housing.

The mass can have a weight of at most about 50 g, 40 g, 30 g, 25 g, 20 g, or 15 g.

The mass can have weight of at least about, 1 g, 2 g, 5 g, or 10 g.

In embodiments, the mass is preferably between 2 g and 20 g.

The weight of the mass can depend on the application. In other words, the vibration generator can be adapted to an application by comprising a mass that is optimized for this application.

An amplitude of the oscillatory motion can be at most about 50 mm, 45 mm, 40 mm, 35 mm, 30 mm, 25 mm, 20 mm, 15 mm, 10 mm, 5 mm, or 2 mm. The amplitude can depend on the application. In other words, the amplitude can be adapted to an application. For example, the amplitude can be below 5 mm, in particular below 2 mm for treatments of the paranasal sinuses of a human being.

For example, the amplitude can be 1 mm, 2 mm, 3 mm, 4 mm, or 5 mm.

The vibration generator may be configured for oscillations of at least about 1 Hz, 5 Hz, 10 Hz, 20 Hz, 30 Hz, 40 Hz, 50 Hz, 60 Hz, 70 Hz, 80 Hz, 90 Hz, or 100 Hz.

The vibration generator may be configured for oscillations of at most about 2000 Hz, 1900 Hz, 1800 Hz, 1700 Hz, 1600 Hz, 1500 Hz, 1400 Hz, or 1300 Hz.

The vibration generator can be configured for oscillations preferably in the range of 1 Hz to 2000 Hz, more preferably in the range of 20 Hz to 1500 Hz, and more preferably in the range of 60 Hz to 1300 Hz. In other words, the oscillations are preferably in the range of 1 Hz to 2000 Hz, more preferably in the range of 20 Hz to 1500 Hz, and more preferably in the range of 60 Hz to 1300 Hz. Typically, the oscillations sweep in a range of at least about 60 Hz to at most about 1300 Hz.

It is an insight that the vibration generator disclosed is in particular suitable to work in the range of 60 Hz to 1300 Hz because the amplitude of an oscillatory motion of a vibration generator of the kind described increases with decreasing frequency. Further, the amplitude and frequency of the oscillatory motion of a vibration generator of the kind disclosed can be well controllable in said range. In particular, amplitude and frequency can be better controlled in comparison to alternative vibration generators.

One can envisage various shapes of the permanent magnet, such as ring-, disc-, or square shape.

The permanent magnet can comprise Neodymium, for example. In other words, it can be a so-called Neodymium magnet.

In an embodiment the permanent magnet is a ring magnet, wherein the ring magnet and the coil are arranged concentrically around the axle. For example, the ring magnet and the coil can be arranged concentrically around the axle, wherein the coil is arranged closer to the axle than the ring magnet.

The ring magnet and the coil can be offset along the axle.

The vibration generator can comprise a plurality (i.e. two or more) of ring magnets. In this case, the ring magnet mentioned before can be considered as a first ring magnet.

The further ring magnet(s) can be arranged concentrically with respect to the axle, too.

The further ring magnet(s) can have the same dimensions as the first ring magnet, and it/they can be offset along the axle. For example, a further ring magnet can be offset along the axle and can be adjacent to the first ring magnet.

The number and arrangement of the further ring magnet(s) can be such that the magnetic field, in particular the magnetic field strength and/or the magnetic field distribution, is optimized with respect to the mass used and/or desired treatment.

In an embodiment, the mass comprises a slit and the slit is concentrically with respect to the axle, too.

In this embodiment the coil can be arranged in the slit. This also means that the slit is or comprises an annular aperture (a ring-shaped opening) that is closer to the axle than the first and—as the case may be—at least one further ring magnet.

In an embodiment, the mass and the ring magnet (or ring magnets) are configured to generate an essentially homogeneous magnetic field in a section of the slit, wherein the homogeneous field runs radial to the axle in this section of the slit at least.

For example, the section of the slit in which the essentially homogeneous field is generated can be formed by a portion of the mass forming a core around which the ring magnet (or ring magnets) is arranged and a core ring. The portion of the mass comprising said core is called “core bottom” in the following.

The core ring can be arranged with respect to the ring magnet(s) and the core bottom in a manner that the essentially homogenous field is generated.

The core ring can comprise or can be of a material, in particular of a metal, that is well suited to conduct magnetic fields. In particular, the material can have a high saturation level, for example a saturation level that is greater than 1 T or greater than 1.5 T. The dimensions of the core bottom can be such that a saturation limit of the material (metal) in regard to magnetic field is not exceeded. Thus, the core bottom and core ring act in effect as guides for the magnetic flux resulting in the essentially homogeneous magnetic field in the section of the slit.

In an embodiment, an extension of the coil in a direction parallel to the axle, this means a length of the coil, is smaller than an extension of the section comprising the homogeneous field, said extension of the section being in a direction parallel to the axle, too.

In this embodiment, the vibration generator is configured such that the coil is in the section comprising the homogeneous field independent of the orientation of the vibration generator.

In particular, it is in the section comprising the homogeneous field in an idle state of the vibration generator, this means in a state in which no current flows in the coil.

The vibration generator can further be configured for the oscillatory motion of the mass being restricted between two positions of maximum deflection of the mass and for the coil being predominantly in the section of homogeneous field.

In particular, the coil can be predominantly in the section of homogeneous field independent of the position of the mass between the two positions of maximum deflection.

A homogeneous magnetic field, in particular in combination with a coil as disclosed that oscillates in the homogenous field only or predominantly is important to have a consistent and well controllable response of the movement of the mass to current generated in the coil.

In an embodiment, the extension of the coil in a direction parallel to the axle is greater than the extension of the section in a direction parallel to the axle.

In this embodiment, the vibration generator is configured such that a portion of the coil extends over the full extension of the section independent of the position of the mass.

Again, the oscillatory motion of the mass can be restricted between two positions of maximum deflection of the mass.

An embodiment having the coil with an extension that is greater than the related extension of the section has the advantage of a maximum number of windings in the section and independent of the position of the mass, for example. This is advantageous in terms of actuation of the mass, such as actuation force.

In an embodiment, the vibration generator comprises at least one elastic element that centers the mass when the vibration generator is not powered.

In particular, the at least one elastic element centers the mass in a manner that the coil is arranged in the slit, in particular in the section of the slit comprising the essentially homogeneous field.

In embodiments, the vibration generator comprises two elastic elements, for example two elastic elements arranged around or in proximity to the physical axle.

In an embodiment, the at least one elastic element is compressed during oscillation of the mass.

The elastic element or a plurality of elastic elements can be configured to delimit the amplitude of the oscillation.

The elastic element(s) can be configured to delimit a maximal deflection of the mass. In particular, the elastic element(s) can define the two positions of maximum deflection.

Alternatively, the elastic element(s) can be configured such that a stop or a plurality of stops delimit the maximal deflection of the mass. For example, the stop can be given by a bearing of the elastic element(s), such as the housing and/or a coil bracket.

The elastic element can be a spring, in particular a coil spring.

For example, the vibration generator comprises two elastic elements, wherein one delimits the deflection (amplitude) of the mass in one direction along the axle and the other one delimits the deflection of the mass along the other direction along the axle.

The mass can be suspended by the two elastic elements that may be a coil spring.

The vibration generator can be configured that no harmonics, in particular no harmonics of significance with respect to the amplitude of the oscillatory motion of the mass at least, are in the frequency range used for the treatment. In other words, preferably the treatment frequency range is different to the resonance frequency of the device itself.

This can be done by coordinating the elastic properties of the elastic element and the weight of the mass, for example.

In particular, the vibration generator can be configured that at least the first (basic) harmonic is outside, in particular below, the frequency range used for treatment.

A vibration generator that is configured to have no harmonics or at least no harmonics of significance with respect to the amplitude in a determined frequency range is advantageous in combination with applications comprising a sweep over a frequency range.

For example, the vibration generator or a device comprising the vibration generator can be configured to operate off-resonant. This means that the vibration generator or device can be configured to omit or pass rapidly through frequencies or frequency ranges corresponding to harmonic frequencies.

In an embodiment, the coil is mounted on a support having good heat transfer properties, wherein the support is in thermal connection to a housing of the vibration generator. The housing is of a material capable to absorb heat generated by the coil and transferred to the housing via the support.

For example, the specific thermal capacity of the housing and/or the support can be larger than 400 J/kg⁻¹ K⁻¹. The housing and/or the support can comprise or consist of steel.

For example, the specific thermal capacity of the housing and/or the support can be greater than 900 J/kg⁻¹ K⁻¹. The housing and/or the support can comprise or consist of aluminium.

In an embodiment, the vibration generator or a device comprising the vibration generator can comprise a signal processing unit, wherein the signal processing unit is configured to superimpose a control signal, this means the input signal used for generating the movement of the mass, with a further signal.

The further signal and the vibration generator can be configured in a manner that an audio signal can be generated from the further signal.

The vibration generator can be configured to sweep over a plurality of frequencies. For example, the vibration generator (or a device comprising the vibration generator) can comprise a controller configured to run the vibration generator in a manner comprising a sweep.

With respect to medical applications, there are indications that a sweep can improve treatment efficiency by exciting a plurality of resonances, also resonances of different kinds as disclosed in relation to the application example below, for example.

The resonance frequencies can be subject-specific. The sweep can also be configured to make sure that at least one resonance frequency is in the applied range of frequencies independent from the stimulated subject.

For example, the vibration generator or the device comprising vibration generator can be configured to sweep over the frequency range of 60 to 1300 Hz or a section of it.

The sweep over a plurality of frequencies can be characterised by a sweep time, this means by the time needed for scanning from the lowest frequency value of the plurality of frequencies to the largest frequency value and back to the lowest value.

The sweep time can be at most about 60 s, 45 s, 30 s, 25 s, 20 s, 15 s, 10 s, or 5 s. The sweep time can be at least about 0.5 s, 1 s, 1.5 s, 2 s, 3 s, 4 s, or 5 s.

The sweep time is preferably between 0.5 s and 30 s, more preferably between 1 s and 10 s.

The plurality of frequencies can be given by any frequency range disclosed above.

The sweep time can vary during operation of the vibration generator. In other words, a sweep rate can vary. In particular, the sweep time can vary during operation within any time range that arise from the sweep times disclosed above. For example the sweep time can vary between 0.5 s and 30 s or between 1 s and 10 s.

For example, the sweep time can decrease during operation. In other words, the sweep rate can increase. A decreasing sweep time (increasing sweep rate) can have the benefit of an increasing energy transfer from the vibration generator to a subject in contact with the vibration generator.

The vibration generator or the device comprising the vibration generator can be configured to carry out a plurality of sweeps during operation.

The invention relates further to a device for applying oscillations (vibrations) to a subject to be stimulated, wherein the device comprises a vibration generator in any embodiment disclosed.

It has been found that a vibration generator as disclosed comprises various benefits that make the vibration generator suitable for being used in oscillation (vibration) therapy:
The vibration generator comprising a coil, in particular a coil as disclosed (and sometimes called a voice coil), can have properties that make such a vibration generator very suitable for use in the field of oscillation (vibration) therapy in comparison to a piezoelectric transducer or a transducer comprising a rotation mass, for example.
   For example, such a vibration generator can generate vibrations that are directed or even focused in a direction.
The vibration generator can be designed to have an amplitude of the vibrations that is more homogeneous over the whole frequency range of interest in the field of oscillation (vibration) therapy compared to piezoelectric transducers or a transducer comprising a rotation mass, for example. This is one reason why the vibration generator can be well suited for the frequencies at the upper end of the frequency range of interest.
   In particular, a design in which the mass comprises the magnets leads to a heavier mass and allows for higher intensities without increasing space requirements and without increasing the overall weight of the vibration generator. It further allows for a more homogeneous magnetic field in the actuation region of the vibration generator without increasing space requirements and without increasing the weight of the vibration generator. A more homogeneous magnetic field in the actuation region leads to a more linear response of the vibration generator to the electric input signal and to a more homogeneous amplitude over the frequency range of interest, for example.

In particular, the device can be configured for the treatment of paranasal sinuses, for example for the treatment of chronic rhino sinusitis.

In an embodiment, the device comprises a device head and optionally a device body, wherein the vibration generator is arranged in the device head.

The device body can be designed for being held by a user.

The device can be a handheld device.

The device can be portable.

The device can be configured for a drug free use.

The device can be configured for a non-invasive use.

The device, in particular the device head, can be designed to comprise a surface (called “contact surface” in the following) that can be brought in contact to the subject, for example when the device is held at the device body and when the device is in a state suitable for stimulation of the subject.

The device can be configured for direct contact between the surface and the subject, this means between the surface and the skin of the body portion to which the device is applied, during use. In other words, there is no need for an intermediate element or layer between the surface and the skin. In particular, there is no need for a gel and the like.

In an embodiment, the device head is movable to a first position relative to the device body and to a second position relative to the device body.

The device comprises further a controller configured to switch the device in a sleeping mode if the device head is moved to the first position and to switch the device in an active mode, if the device head is moved to the second position.

In an embodiment, the device head is in addition movable to a third position relative to the device body, wherein the third position allows access to the contact surface for cleaning.

In this embodiment, the controller is configured further to switch the device in the sleeping mode if the device head is moved to the third position.

For example, the device body can comprise a recess and the device head can be designed in a manner that it can be stored completely in the recess. In particular, the device head can be flush with the device body.

In this case, the position of the device head in which it is stored completely in the recess can be the first position.

In this case, the first position can also be considered as a closed position.

A device head being in the closed position is prevented from at least one of contamination, unintentional start and damage, for example.

The device can be equipped for the device head being moved out at least partly of the recess.

For example, the device can comprise an axis around which the device head can be pivoted or along which the device head can moved.

If the device comprises the axis around which the device head can be pivoted and if a rotation angle of 0° corresponds to the first position (closed position, device in sleeping mode), the second position (active mode) can be at a rotation angle between 90° and 150° degrees, for example. For example, the second position can be between 110° and 130°, such as at 115°, 118°, 120°, 122° or 125°.

In particular, the second position can be at most about 150°, 145°, 140°, 135°, or 130°. The second position can be at least about 90°, 95°, 100°, 105°, or 110°.

In such configurations, the optional third position (cleaning mode) can be at a rotation angle between 150° and 200° degrees, for example. For example, the third position can be at 160°, 170°, 180°, or 190°.

In an embodiment, the third position is at 180°.

The device can comprise fixation means that allow automatic or manual fixation of the device head relative to the device body in at least one position.

The device can be configured to move the device head to at least one of the first, second or third position in an automated manner.

Alternatively or in addition, the device can be configured to move the device head between at least two of the first, second and third position in an automated manner.

The device can comprise a motor, in particular an electric drive, configured to move the device head in an automated manner.

Alternatively or in addition to an automated movement of the device head, the device can be configured to move the device head manually.

In the sleeping mode and—if present—the cleaning mode, the vibration generator can be inactive.

The device can be configured to start a stimulation in an automated manner, in particular to activate the vibration generator, in case the device head is moved to the second position.

The invention relates further to a method for treating a subject with oscillations. The method comprises a step of bringing a device comprising a vibration generator in any embodiment disclosed or a device for applying oscillations in any embodiment disclosed in contact with the subject.

The method can comprise a step of treating the subject during a treatment time of at least about 0.5 s, 1 s, 2 s, 5 s, 10 s, 15 s, or 20 s.

The method can comprise a step of treating the subject during a treatment time of at most about 5 min, 4 min, 3 min, 2 min, 90 s, 60 s, 45 s, or 30 s.

The treatment time can be between 0.5 s and 2 min, for example between 2 s and 90 s, 2 s and 60 s, or 5 s and 30 s. For example, it can be 5 s, 10 s, 15 s, 20 s, 25 s, 30 s, 45 s, 60 s, 75 s, 90 s, 105 s, or 120 s.

The method can comprise a step of carry out the treatment a plurality of times. In other words, the method can comprise a plurality of treatment sessions.

The subject matter of the invention will be explained in more detail in the following text with reference to exemplary embodiments which are illustrated in the attached drawings, which schematically show:

FIG. 1 shows an exterior view of an exemplary embodiment of a device 1 for applying oscillations (vibrations) to a subject by comprising a vibration generator 10.

The device 1 shown is a compact, handheld device.

The device comprises a device body 2 and a device head 3, wherein the device head 3 hosts the vibration generator 10.

The device head 3 shown comprises a contact surface 4 that is arranged to be brought at least partly in contact with the subject to be treated.

The contact surface 4 may be a surface of an interchangeable part 5 of the device 1

In the embodiment shown, the contact surface 4 comprises an indentation 7 in the shape of a convex recess.

The device head 3 shown is pivoted with respect to the device body 2 (indicated by a double arrow). The pivotal mounting can be such that the device head 3 can be brought at least in the first and second positions relative to the device body 2 mentioned above. In addition, the device head 3 can be optionally brought at least in the third position mentioned above.

In other words, the device head 3 shown is a movable device head.

The device shown comprises further user interface 26 comprising a plurality of LEDs. The LEDs can indicate at least one of a status of the device and a status (advancement) of a treatment or a session of treatments.

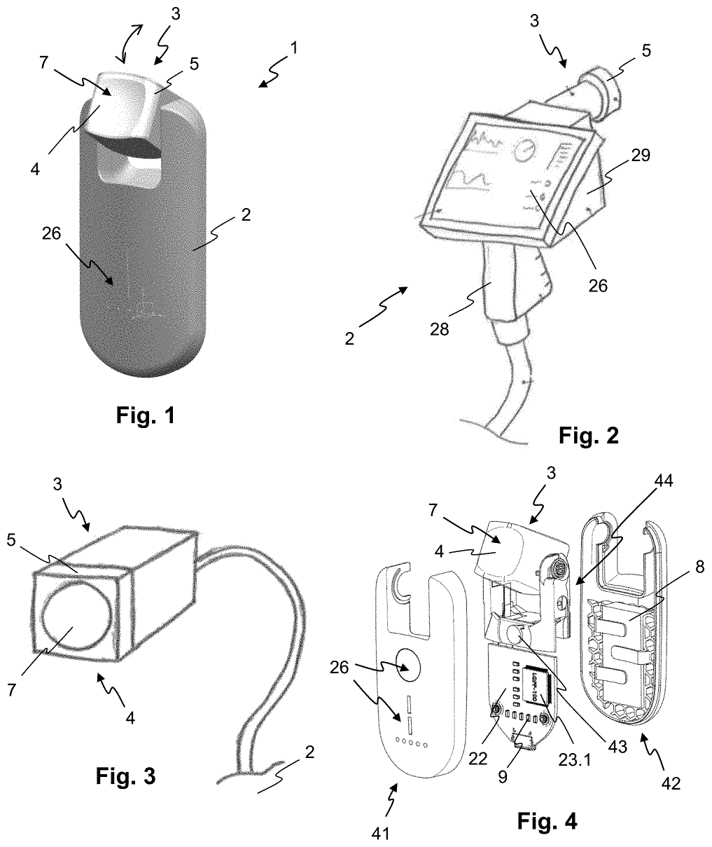

FIG. 2 shows an external view of a further exemplary embodiment of a device 1 comprising the vibration generator 10.

The device 1 shown may be handheld, however it is not as compact as the device 1 of FIG. 1. In other words, the device 1 of FIG. 2 is rather suitable for being installed in or provided by hospitals and professionals whereas the device 1 of FIG. 1 is rather suitable for use by a wider public and can be carried around by a user, for example.

The device 1 of FIG. 2 comprises—in comparison with the device 1 of FIG. 1 at least—a powerful computerized device 29 and a more detailed user interface 26. Optionally, it can comprise a fixture or grip 28.

FIG. 3 shows mainly an external view of an exemplary embodiment of a device head 3 comprising the vibration generator 10.

The device head 3 shown is a handheld device head 3, wherein the device body 2 can be handheld, for example a cell phone or a tablet, or firmly installed, such as a personal computer (PC) or another computerized device, e.g. as shown in FIG. 2.

The device body 2 can supply the device head 3 with power and/or control signals, for example. In the embodiment of FIG. 3, such supply is carried out by a wired connection between device head 3 and device body 2.

FIG. 4 shows an exploded view of the device 1 shown in FIG. 1.

In the embodiment shown, a housing of the device body comprises a front part 41 and a rear part 42.

The rear part 42 is equipped to hold a battery 8, for example a rechargeable battery.

Front and rear part are designed to host the more sensitive parts of the device 1, such as a Printed Circuit Board (PCB) 22, a controller 23.1 of the device 1, components of the user interface 26, such as LEDs 9 and at least one manual control element 43 (control knob, button etc.), and at least one support 44 for the device head 3, which is a movable device head in the embodiment shown.

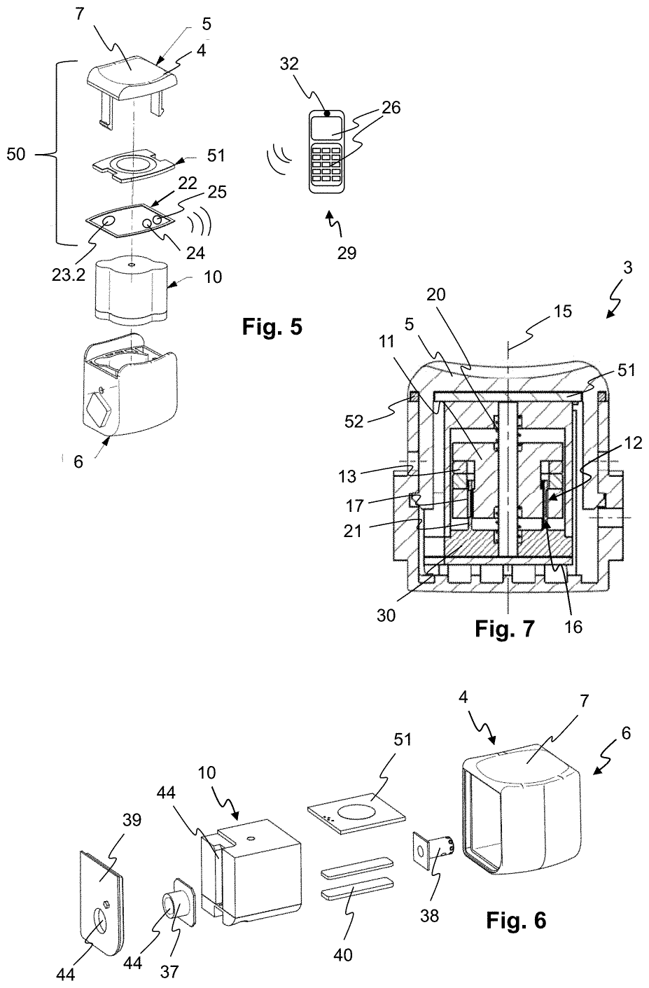

FIG. 5 shows an exploded view of an exemplary embodiment of the device head 3 shown in FIG. 1.

The shape of the device head 3 is given by a housing 6 and the interchangeable part 5.

The interchangeable part 5 can be mounted to the housing 6 by comprising a protrusion arranged on the interchangeable part 5 to reach into the housing 6 and designed to form a positive-fit connection with the housing 6, for example.

The device head 3 shown comprises further a capacitive (touch) sensor 51, a transducer (vibration generator) 10, and a Printed Circuit Board (PCB) 22.

In the embodiment shown, the interchangeable part 5 comprising the contact surface 4 and the indentation 7, the capacitive sensor 51 and the PCB 22 are the main components of a sensor element 50 configured to detect a contact between the contact surface 4 and the subject and to generate a related output signal.

The output signal is pressure dependent in some embodiments.

The PCB 22 comprises a controller 23.2 of the sensor element configured to generate the output signal.

The PCB 22 can further comprise a memory 24 and/or communication means 25 to a computerized device 29.

In an alternative embodiment, the capacitive sensor 51 comprises the controller 23.2, the memory 24 and/or the communication means 25.

The communication means 25 can be wireless communication means or wired communication means, as it is the case in the embodiments of FIGS. 2 and 3, for example.

The computerized device 29 can be a handheld (portable, mobile) computerized device, such as a cell phone or a tab, or it can be a firmly installed computerized device as disclosed with respect to FIGS. 2 and 3.

The computerized device 29 can comprise a user interface 26 and can be configured to run an application (program) suitable for at least one of controlling the device 1, comparing a characteristic of the output signal with a present value, determining whether the characteristic of the output signal is greater than a pre-set value, generating an enable signal, setting a timestamp when a treatment is started, determining a treatment regularity, determining a treatment completeness, determining a contact quality, determining a treatment quality, selecting a desired treatment, and indicating the target position and optionally the target orientation, for example.

The treatment regularity expresses whether a sequence of treatments is carried out with a regularity needed for a given application. For example, the treatment regularity can be determined by comparing a period between two timestamps with a pre-set period, wherein the pre-set period can be an optimal period between two treatments for a specific treatment.

The treatment completeness expresses whether the number of treatments carried out is sufficient for a given application. For example, the treatment completeness can be determined by comparing a number of timestamps set during a period (e.g. a day or a week) in which the overall treatment is planned to take place time with a pre-set number of treatments.

The contact quality expresses whether the contact between the device 1 and the subject to be stimulated is sufficient during a treatment for a given application. For example, the contact quality can be determined by determining whether the characteristic of the output signal is greater than a pre-set value repeatedly during a treatment and by setting the number of characteristics greater than the pre-set value in relation to the total number of output signals analyzed.

The treatment quality expresses whether a treatment is carried out on the subject under conditions that result in a good treatment. For example, the determination of the treatment quality can comprise reading out the pressure dependent output signal, in particular the value of the characteristic that is related to the contact pressure, repeatedly during a treatment and setting the read-out pressure dependent output signals in relation to a target value. The target value may be a time-dependent target value.

For example, the determination of the treatment quality can comprise determining a ratio between an integral of the time evolution of the read-out pressure dependent output signal and an integral of the time evolution of a target pressure. In this case, a ratio lager than 1 can be considered as a good contact quality leading to a good treatment quality, for example.

For example, the determination of the treatment quality can comprise determination of a percentage. For example, a good treatment quality can be assumed if the read-out pressure dependent output signal is greater than the target value during at least 50% of the treatment time, in particular during at least 60%, at least 70%, at least 80% or at least 90% of the treatment time. In other words, a good treatment quality is ensured if the pressure applied during the treatment time is above a pressure threshold value during at least 50%, at least 60%, at least 70%, at least 80% or at least 90% of the treatment time.

The controller 23.2 in combination with the memory 24 and/or user interface 26 as the case may be can be configured to carry out one, a plurality or all of the actions listed above. One, a plurality or all of the actions listed above can be carried out by the controller 23.1 of the device 1.

The controller 23.2 of the sensor element can be integrated in the controller 23.1 of the device 1. The memory 24 and/or the communication means 25 can be arranged on a device PCB 22 as shown in FIG. 4, for example.

FIG. 6 shows an exploded view of a further exemplary embodiment of a device head 3.

In the embodiment shown, the contact surface 4 comprising the indentation 7 is an integral part of the housing 6 of the device head 3.

Due to this, the design of some components of the device head 3 is different compared to the device head 3 according to FIG. 5. For example, the device head 3 comprises a cover plate 39 for closing the device head 3 after arranging the sensor element 50 and the vibration generator 10 in the housing 6.

The exploded view of FIG. 6 shows further bearing (37, 38) for a pivotal mounting of the device head 2 and ducts 44 for wires. A duct in the cover plate 39, a duct in a bearing 37, and a duct on the vibration generator 10 is visible in the exemplary embodiment of FIG. 6.

The exploded view of FIG. 6 shows further buffers 40, for example rubber buffers.

FIG. 7 shows a sectional view of the (assembled) device head 3 of FIG. 5. Among other things, details of the vibration generator 10 and the positive-fit connection between the interchangeable part 5 and the housing 6 are shown.

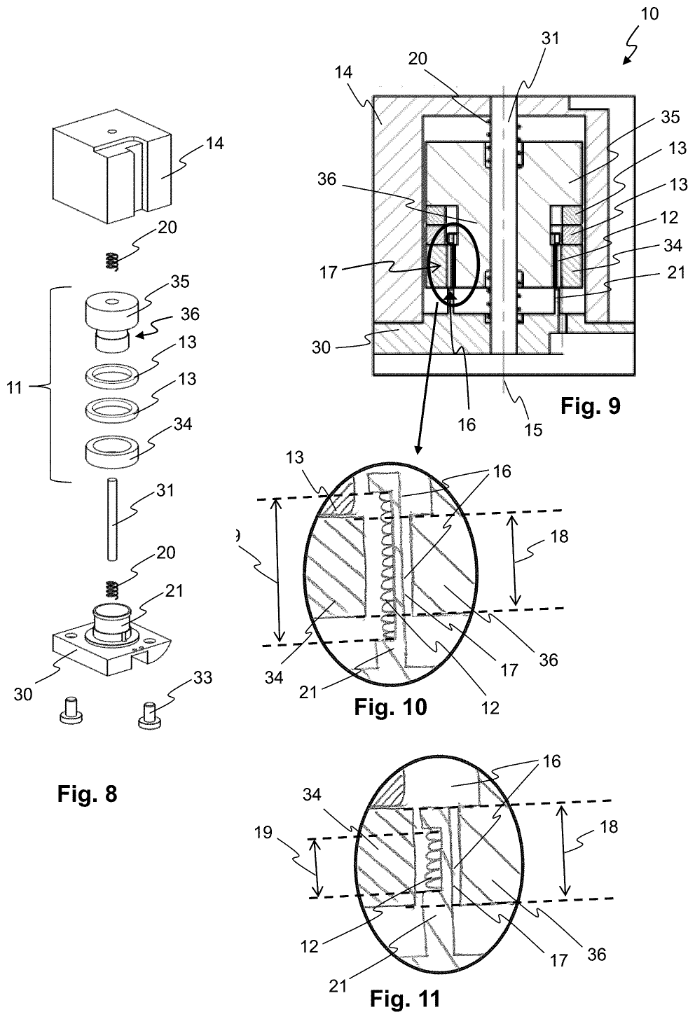

Details of an exemplary embodiment of the vibration generator 10 are disclosed with respect to FIGS. 8 to 11:

FIG. 7 shows a gasket 52 for sealing an interior of the device head 3 in addition to the components shown in FIGS. 5 and 8-11.

FIG. 8 shows an exploded view of an exemplary embodiment of a vibration generator 10.

The shape of the vibration generator is given by a housing 14 of the vibration generator and a so-called coil bracket 30.

The vibration generator 10 comprises further a (physical) axle 31 defining a (directional) axis 15, a permanent magnet (two ring magnets 13 in the embodiment shown), a so-called core ring 34, a so-called core bottom 35, and a coil 12 (not shown in FIG. 11), in particular a coil as disclosed in the following. A coil as disclosed in the following is sometimes called a voice coil 12.

The coil bracket 30 can be considered as a base of the vibration generator 10, said base comprising a support 21 for the coil 12.

The mass 11, this means the component of the vibration generator 10 that can be actuated to carry out an oscillatory motion along the axis 15, comprises the core bottom 35, the permanent magnet (the ring magnets 13 in the embodiment shown) and the core ring 34.

The core bottom 35 can account for most of the weight of the mass 11. The weight of the core bottom 35 can be adjusted to the application.

The vibration generator 10 comprises further two elastic elements (coil springs 20) in the embodiment shown. The springs 20 are configured to center the mass and to generate a repelling force to the mass 11.

In the embodiment shown, it is the housing 14 and the coil bracket 30 that delimit the maximum deflections of the mass 11 by the elastic elements (coil springs 20) being partly arranged in a recess of the core bottom and the coil bracket 30, respectively.

FIG. 9 shows a sectional view of the assembled vibration generator of FIG. 8.

In the embodiment shown, one end of the axle 31 is mounted to the coil bracket 30 and the other end of the axle 31 is mounted to the housing 14.

A first spring 20 is arranged around the axle 31 at its mounting point to the housing 14 and a second spring 20 is arranged around the axle 31 at its mounting point to the coil bracket 30.

The housing 14 and first spring 20 as well as the coil bracket 30 and the second spring 20 define maximal deflections of the mass.

The coil bracket 30 is mounted to the housing 14, for example by screws 33.

In the embodiment shown, the core bottom 35, the ring magnets 13 and the core ring 34 are arranged concentrically with respect to axis 15.

Further, the core bottom 35, the ring magnets 13 and the core ring 34 are firmly mounted to each other, for example by gluing. In other words, the mass 11 is formed integrally (one-piece).

The core bottom 35 comprises a protrusion 36, wherein the ring magnets 13 and the core ring 34 are arranged around the protrusion 36.

The protrusion 36 is designed for forming a slit 16 between the protrusion 36 and the core ring 34. The slit 16 runs concentrically with respect to the axis 15.

The protrusion 36 can be designed further for the slit 16 being formed between the ring magnets 13 and the protrusion 36, too.

The core ring 34 and a portion of the protrusion 36 that forms the slit 16 between the core ring 34 and the protrusion 36 can be designed for an optimized magnetic field in a section 17 of the slit 16 formed by the core ring 34 and the protrusion 36.

The magnetic field is optimized in terms of homogeneity, for example.

In the embodiment shown, the magnetic field lines run (or rather have to run) radial to the axis 15 in said section 17 of the slit 16.

The support 21 and the coil 12 held in position by the support 21 are designed for extending into the slit 16 in a manner that at least a portion of the coil 12 is arranged in the section 17 of the slit 16 formed by the core ring 34 and the protrusion 36. In particular in the idle state of the vibration generator 10, at least a portion of the coil 12 is in said section 17.

FIG. 10 shows a detail view of the coil 12, the coil ring 34 and the protrusion 36 in the section 17 of optimized magnetic field, this means in an actuation region of the vibration generator 10.

In the embodiment shown, an extension 18 of the section 17, said extension 18 being parallel to the axis 15, is smaller than a related extension 19 of the coil 12.

In particular, the extension 19 of the coil 12 is such that a portion of the coil 12 extends over the full extension 18 of the section 17 independent of the position of the mass 11.

As shown with respect to FIG. 9, the position of the mass 11 is within two positions of maximal deflection.

A configuration between the coil 12 and the section 17 as shown in FIG. 10 has the advantage of a maximum number of windings being always within the actuation region. This is advantageous in terms of actuation of the mass, such as actuation force.

FIG. 11 shows a detail view of an alternative actuation region.

In the embodiment shown, the extension 18 of the section 17 is larger than the related extension 19 of the coil 12.

In particular, the extension 19 of the coil 12 is such that the whole coil 12 is within the section 17 of optimized magnetic field at least in idle state but independent of the orientation of the vibration generator 10.

Optionally, the whole coil 12 is within the section 17 of optimized magnetic field independent of the position of the mass 11.

A configuration between the coil 12 and the section 17 as shown in FIG. 11 has the advantage of the coil 12 being in region of homogeneous magnetic field only. This is advantageous in terms of response behaviour of the mass 11 and controllability of the oscillatory motion of the mass, for example.

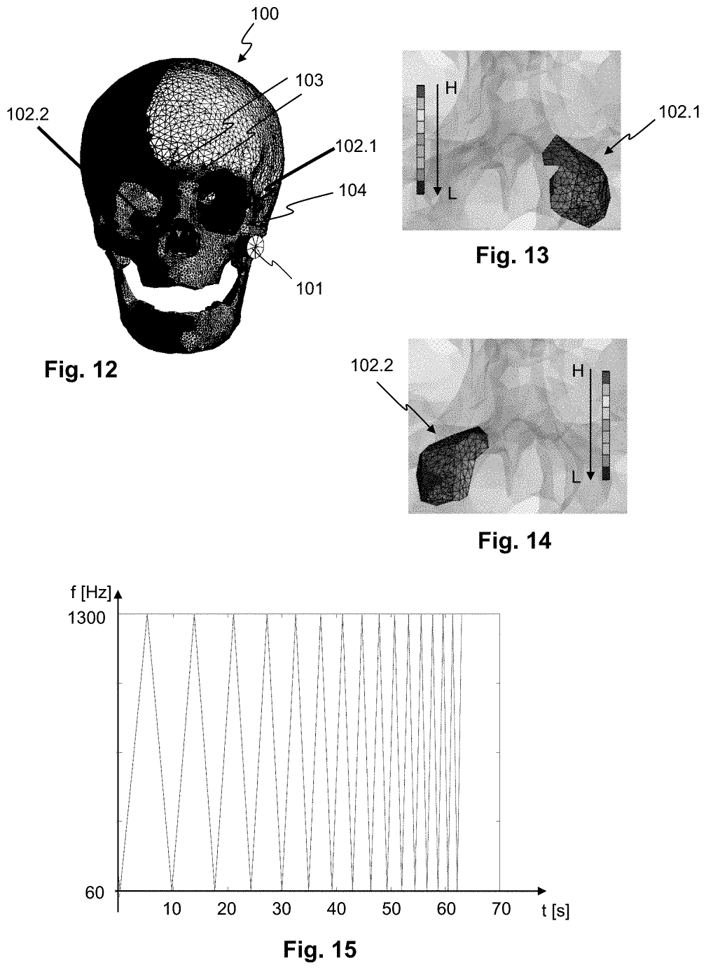

FIGS. 12-15 show an application example of the vibration generator and the device, namely the treatment of chronic rhinosinusitis (CRS) by modulated vibration therapy and by use of a device 1 as shown exemplarily in FIGS. 1 and 4 and comprising a vibration generator 10 as shown exemplarily in FIGS. 8-10.

FIG. 12 shows a model of a human skull. The human skull (more precisely the human head) is the subject 100 in the application example. Such a model of the human skull was used to carry out numerical simulation with the aim to get information about the mechanical, in particular vibrational, properties of the human head and to supply indications of the vibrational excitation of the maxillary sinuses (left maxillary sinus 102.1, right maxillary sinus 102.2) and of the frontal sinuses 103.

The sinuses cannot be seen in FIG. 12 because they are arranged inside the skull (mainly behind maxilla and frontal bone, respectively).

FIG. 13 visualizes a numerically calculated deformation of the left maxillary sinus 102.1 when excited by vibrational energy with a frequency close to a numerically calculated resonant frequency of the maxillary sinus and when the vibrational energy is coupled into the skull by a vibration generator at the application point 101, this means by a vibration generator in contact with the zygomatic bone 104 at the indicated application point 101.

The colours are indicative for the degree of deformation, wherein the colour next to H indicates a high deformation and the colour next to L indicates a low deformation.

FIG. 14 visualizes a numerically calculated deformation of the right maxillary sinus 102.2 when excited as discussed in relation to FIG. 13. This means, an effect on the right maxillary sinus 102.2 when the vibrational energy is coupled into the left zygomatic bone 104 is shown.

Again, the colours are indicative for the degree of deformation, wherein the colour next to H indicates a high deformation and the colour next to L indicates a low deformation.

FIGS. 13 and 14 show snapshots of the deformation of the maxillary sinuses due to the vibrational energy coupled into the left zygomatic bone 104, only. The time-dependent deformation of the maxillary sinuses is an oscillating deformation between the deformation states shown in FIGS. 13 and 14 and an opposite state.

FIGS. 13 and 14 suggest that the maxillary sinuses can be excited to oscillating deformation by vibrational energy of a specific frequency, i.e. a resonant frequency of the maxillary sinuses, applied to the zygomatic bone 104.

FIGS. 13 and 14 suggest further that a coupling of vibrational energy into the left zygomatic bone may not only have an effect on the left maxillary sinus 102.1 but also on the right maxillary sinus 102.2, and vice versa.

The frequency of the vibrational excitation resulting in FIGS. 13 and 14 was around 355 Hz. However, the numerical simulations suggest various further resonance frequencies between 100 Hz and 1300 Hz, at least.

The numerical simulations carried out supply indications of the structure-mechanical properties of a sinus. Another aspect of the vibrational properties of a sinus can be obtained by approximating the sinus by a Helmholtz resonator and by using the Helmholtz equation to estimate air resonances in the cavity formed by the sinus (by the Helmholtz resonator). A basic resonance frequency of around 27.6 Hz for the sinuses shown in FIGS. 13 and 14 can be calculated from the Helmholtz equation.

Hence, the numerical simulations and the Helmholtz equation suggest that there are resonances of both structure-mechanical and geometrical kind in a frequency range between 20 Hz and 1300 Hz at least, wherein the structure-mechanical resonances can be excited by the device 1 applied to the zygomatic bone 104. Further, it is conceivable that the vibrations applied to the zygomatic bone 104 can excite the resonances of geometrical kind (i.e. the Helmholtz resonances) via deformation of the sinus if the sinus can be approximated by a Helmholtz resonator.

In principle, it is conceivable that an excitation of structure-mechanical and geometric resonances have a synergetic effect, for example by the structure-mechanical resonance(s) opening the ostium of the sinus and enable the appearance of geometric resonance(s).

However, excitation frequencies below 60 Hz are preferably avoided due to possible adverse effects.

Further, literature suggests resonant frequencies of the frontal sinuses between 160 Hz and 1240 Hz.

In summary, a frequency range between 60 Hz and 1300 Hz is a preferred frequency range for the treatment of CRS.

Scanning over a frequency range, for example over the preferred frequency range, guarantees that the sinuses are excited at various resonant frequencies and it guarantees further that subject dependent variations of the resonant frequencies of the sinuses do not have an adverse effect on treatment success.

The influence of sweep time, this means the time for scanning from the lowest frequency value of the preferred frequency range to the largest frequency value and back to the lowest value, on energy transmission from the device 1 to the subject 100 was estimated experimentally. The experiments indicate an increased energy transmission for small sweep times, in particular for sweep times below 5 seconds, whereas the energy transmission is essentially constant for sweep times between 5 and 30 seconds, at least.

In other words, low sweep times seem to be preferable in terms of efficient energy transmission from the device 1 to the subject 100. However, low sweep times are often found unpleasant by the user (patient). Further, the influence of sweep time on the excitation efficiency of the sinuses has to be studied further yet.

Hence, a sweep time that changes during a single treatment seems to be advantageous. Further, a changing sweep time can be used to make the users perception of the treatment less boring and/or to signal the approaching end of the treatment to the user.

FIG. 15 shows an exemplary course of the vibration frequency produced by the device 1 for CRS treatment. The sweep time decreases from 10 s to 1.5 s. The scanned frequency range is 60 Hz to 1300 Hz.

One can envisage other course of the vibration frequency, for example a course with a constant sweep time and/or sweep time(s) that are within a range given by efficient resonant excitation of a sinus.

A method for treating chronic rhinosinusitis (CRS) by modulated vibration therapy can be as follows when considering the above:
The contact surface 4 of the device 1 is applied to the application point 101 on skin over the left cheekbone of the subject 100.
The device 1 is activated, this means the device generates vibrations in the frequency range between 50 Hz and 1600 Hz, in particular between 60 Hz and 1300 Hz, wherein the frequency range is repeatedly scanned with a sweep time between 0.5 s and 30 s, for example between 1 s and 10 s.
   The sweep time can vary during the treatment. For example, the course of the vibration frequency can be as shown in FIG. 15.
The device 1 is deactivated after a pre-set treatment time. The treatment time can be in the range of 0.5 s to 2 minutes, for example 1 minute, in the case of CRS treatment.
The treatment is repeated on the right cheekbone.
   The treatment time can be longer than the above-disclosed 0.5 s to 2 minutes if the treatment is carried out at one cheekbone, only. In this case, the treatment time can be 2 or 3 minutes or between 2 and 3 minutes, for example.

The method for treating CRS usually comprises a plurality of treatment sessions. This means, the steps listed above are repeated a plurality of times in a given period. In particular, 3 to 4 treatment sessions are carried out per day.

1. A vibration generator comprising a mass, a coil, a permanent magnet and a housing, wherein the mass can be set in an oscillatory motion with respect to the housing by applying a current to the coil, and
the vibration generator further comprising an axle, wherein the oscillatory motion is along the axle, and in that the mass comprises the permanent magnet and the coil is fixed to the housing.

2. The vibration generator of claim 1, wherein the permanent magnet is a ring magnet, wherein the ring magnet and the coil are arranged concentrically around the axle.

3. The vibration generator of claim 1, wherein the mass comprises a slit, wherein the slit is arranged concentrically with respect to the axle and wherein the coil is arranged in the slit.

4. The vibration generator of claim 3, wherein the mass and the permanent magnet are configured to generate an essentially homogeneous field in a section of the slit, wherein the homogeneous field runs radial to the axle in this section of the slit.

5. The vibration generator (10) of claim 4, wherein the section is formed between a core ring and a core bottom, wherein the core ring is of a material having a high saturation level and wherein the core bottom is configured to not exceed a saturation limit of the material having the high saturation level.

6. The vibration generator of claim 4, wherein an extension of the coil in a direction parallel to the axle is smaller than an extension of the section in a direction parallel to the axle and wherein the vibration generator is configured such that the coil is in the section independent of the orientation of the vibration generator.

7. The vibration generator of claim 5, wherein the vibration generator is configured for the oscillatory motion being restricted between two positions of maximum deflection of the mass and wherein the vibration generator is configured for the coil being predominantly in the section of the homogeneous field.

8. The vibration generator of claim 4, wherein an extension of the coil in a direction parallel to the axle is larger than an extension of the section in a direction parallel to the axle, wherein the vibration generator is configured for the oscillatory motion being restricted between two positions of maximum deflection of the mass and wherein the vibration generator is configured for a portion of the coil extending over the full extension of the section independent of the position of the mass.

9. The vibration generator of claim 1, wherein the vibration generator comprises an elastic element that centers the mass when the vibration generator is not powered.

10. The vibration generator of claim 9, wherein the elastic element is compressed during operation of the vibration generator.

11. The vibration generator of claim 1, wherein the vibration generator is configured to have its basic harmonic outside the frequency range in which the vibration generator is operated.

12. The vibration generator of claim 11, wherein the vibration generator is configured to have no harmonics of significance with respect to the amplitude of an oscillatory motion of the mass in the frequency range in which the vibration generator is operated.

13. The vibration generator of claim 1, wherein the coil is mounted on a support having good heat transfer properties, wherein the support is in thermal connection to the housing, and wherein the housing is of a material capable to absorb heat generated by the coil and transferred to the housing via the support.

14. The vibration generator of claim 1, comprising a signal processing unit, wherein the signal processing unit is configured to superimpose a control signal used for the oscillatory motion of the mass with a further signal, wherein the further signal and the vibration generator are configured in a manner that an audible signal can be generated from the further signal by the vibration generator.

15. The vibration generator of claim 1, wherein the vibration generator is configured to sweep over a plurality of frequencies.

16. The vibration generator of claim 1, wherein the vibration generator is configured to oscillate at a frequency of not less than 1 Hz, 5 Hz, 10 Hz, 20 Hz, 30 Hz, 40 Hz, 50 Hz, 60 Hz, 70 Hz, 80 Hz, 90 Hz, or 100 Hz.

17. The vibration generator of claim 1, wherein the vibration generator is configured to oscillate at a frequency of not more than about 2000 Hz, 1900 Hz, 1800 Hz, 1700 Hz, 1600 Hz, 1500 Hz, 1400 Hz, or 1300 Hz.

18. The vibration generator of claim 1, wherein the vibration generator is configured for oscillations in the range of 1 Hz to 2000 Hz, more suitably in the range of 20 Hz to 1500 Hz, and optionally in the range of about 60 Hz to about 1300 Hz.

19. The vibration generator of claim 1, wherein the vibration generator is configured to sweep over a frequency range of about 60 to about 1300 Hz, or a section thereof.

20. The vibration generator of claim 19, wherein the sweep occurs over a time period of at most about 60 s, 45 s, 30 s, 25 s, 20 s, 15 s, 10 s, or 5 s.

21. A device for applying oscillations to a subject to be stimulated, comprising a vibration generator according to claim 1.

22. The device of claim 21, comprising a device head and a device body, wherein the vibration generator is arranged in the device head.

23. The device of claim 22, wherein the device head is movable to a first position relative to the device body and to a second position relative to the device body, wherein the device comprises a controller configured to switch the device in a sleeping mode if the device head is moved to the first position and to switch the device in an active mode, if the device head is moved to the second position.

24. The device of claim 23, wherein the device head is movable to a third position relative to the device body, wherein the third position allows access to a contact surface for cleaning and wherein the controller is configured to switch the device in the sleeping mode if the device head is moved to the third position.

25. A method for treating a subject with oscillations, characterized by comprising a step of bringing a device comprising the vibration generator according to claim 1 in contact with the subject.
